(19) United States
(12) Patent Application Publication
(10) Pub. No.: US 2026/0259173 A1
(43) Pub. Date: Sep. 3, 2026

(54) GAS ANALYSIS DEVICE AND SUBSTRATE PROCESSING SYSTEM COMPRISING SAME

(71) Applicant: Dong-Ho CHA, Gyeonggi-do (KR)

(72) Inventors: Dong-Ho CHA, Gyeonggi-do (KR); Soon-Yong CHA, Gyeonggi-do (KR); Si-Jun KIM, Daejeon (KR)

(73) Assignee: Dong-Ho CHA, Gyeonggi-do (KR)

(21) Appl. No.: 18/994,595

(22) PCT Filed: Jul. 18, 2023
(86) PCT No.: PCT/KR2023/010285

§ 371 Date: Jan. 14, 2025

(30) Foreign Application Priority Data
Jul. 18, 2022 (KR) 10-2022-0088263
Jul. 18, 2022 (KR) 10-2022-0088264
Jul. 18, 2022 (KR) 10-2022-0088265

(51) Int. Cl. G01N 27/623; G01N 27/68

(52) U.S. Cl. CPC G01N 27/623; G01N 27/68

(57) ABSTRACT

The present invention relates to a gas analysis device and a substrate processing system comprising the same, and, more specifically, to a gas analysis device and a substrate processing system comprising the same, which can monitor or diagnose a substrate processing process by analyzing an analyte gas introduced into a process chamber for substrate processing or discharged from an exhaust line of the process chamber. The present invention provides a gas analysis device installed in a substrate processing system, the gas analysis device includes an ionizing unit which ionizes an introduced analyte gas to produce ionized gas; a mass analysis unit for analyzing the mass of the ionized gas received from the ionizing unit; and a vacuum pump coupled to the mass analysis unit to adjust the internal pressure of the mass analysis unit.

### TECHNICAL FIELD

The present invention relates to a gas analysis device and a substrate processing system comprising the same, and more particularly to a gas analysis device and a substrate processing system comprising the same, which can monitor or diagnose a substrate processing process by analyzing an analyte gas introduced into a process chamber for substrate processing or discharged from an exhaust line of the process chamber.

### BACKGROUND ART

Semiconductor or display manufacturing equipment may be provided with a gas analysis device configured to analyze gases by ionizing the gases.

For example, the gas analysis device can analyze a gas generated in a process of manufacturing semiconductors or displays or a gas discharged from an exhaust line (FL) of a process chamber to monitor or diagnose process conditions in real time without affecting the process.

As a typical gas analysis device for monitoring or diagnosing substrate processing processes, there is a self-plasma optical emission spectrometer (SP-OES). (See FIG. 1) Referring to FIG. 1, the SP-OES refers to a gas analysis device for analyzing an analyte gas and is coupled to a substrate processing apparatus 200 including a process chamber defining a processing space for substrate processing, and may include an ionizing unit 11 coupled to an exhaust line FL to ionize an analyte gas, a spectroscopic sensor 12 that detects a spectrum of light emitted from the analyte gas ionized by the ionizing unit 11, a controller 13 that analyzes the analyte gas based on data detected by the spectroscopic sensor 12 and controls operation of the ionizing unit 11 and the spectroscopic sensor 12, and a terminal 14 communicating with the controller 13.

The ionizing unit 11 may include a plasma chamber 11a into which an analyte gas is introduced, an electrode 11b forming an induced electric field inside the plasma chamber 11a, and an RF power source 11c that applies RF power to the electrode 11b.

The exhaust line FL of the substrate processing unit 200 may be provided with a valve V to control a gas flow.

The electrode 11b may be a coil wound around the plasma chamber 11a and may generate an induced electric field inside the plasma chamber 11a, whereby the analyte gas can be ionized and excited to a plasma state.

The plasma chamber 11a may be provided with a light transmissive window 11d.

The spectroscopic sensor 12 (Optical Emission Spectrometer) may be a spectrometer sensor that detects the spectrum of light generated in the plasma chamber 11a through the window 11d.

Components of the analyte gas and the like may be analyzed based on the emission spectrum detected by the spectroscopic sensor 12.

However, since a single element has multiple emission spectra and the spectra of multiple elements can overlap at the same wavelength during discharge of the multiple elements, making accurate analysis difficult, the SP-OES is more disadvantageous in terms of sensitivity and resolution than a mass spectrometer (MS).

Korean Patent Publication No. 10-2008-0019279 relates to a gas monitoring device for analyzing gas species contained within an enclosure through optical emission spectroscopy, which includes a means for generating plasma for monitoring in an internal space of a protrusion connected to the enclosure, at least one sensor for picking up optical radiation emitted from the plasma for monitoring, and an emission spectrum analyzer for collecting and analyzing the light picked up by the sensor. To address problems of deterioration in analysis sensitivity and periodic stoppage of a process for internal cleaning due to deposits on the light transmissive window and in the internal space in which the plasma is created, the invention of the publication employs a field generator to deflect particles and electrons ionized by the plasma away from the sensor that picks up light.

However, since a large amount of gas flows from the enclosure into the internal space of the protrusion, the structure disclosed in the publication fails to completely solve the problem of contamination of the internal space of the protrusion and the window due to various particles merely through generation of a deflection field deflecting the particles from the sensor in long-term use of the monitoring device, and cleaning or replacement of a contaminated component with a new component must be performed after separation of a gas analysis device from a substrate processing apparatus, thereby affecting continuity of the substrate processing process.

In addition, since the prior gas monitoring device employs the spectroscopic sensor to analyze a gas, the spectroscopic sensor is disadvantageous in terms of sensitivity and resolution. When a mass spectrometer (MS) is used instead of the spectroscopic sensor in consideration of sensitivity and resolution, the mass spectrometer (MS) must be operated in a high vacuum environment and requires a high vacuum pump. However, since it is difficult to maintain an appropriate internal pressure capable of keeping plasma stable in the plasma chamber due to a variety of pressure ranges in the process chamber depending on the substrate processing process, it is difficult to apply the mass spectrometer (MS) instead of the spectroscopic sensor to gas analysis in the substrate processing system.

As a gas analysis device using the mass spectrometer (MS) that has advantages in terms of sensitivity and resolution, there is an inductively coupled plasma-mass spectrometer (ICP-MS). The IPC-MS refers to a device for measuring and analyzing an ion/charge ratio using the mass spectrometer (MS) in an ionization process in which a liquid sample is sprayed into an aerosol state and introduced into high temperature Ar plasma generated using an ICP torch, and has advantages in terms of sensitivity and resolution. However, the IPC-MS is not suitable for long-term continuous use or for monitoring or diagnosing the substrate processing process.

Another gas analysis device for monitoring or diagnosing a typical substrate processing process is a residual gas analyzer (RGA). (See FIG. 2) The RGA refers to a quadrupole mass spectrometer with a typical mass range of 1 amu to 100 amu or 1 amu to 200 amu, which can be used to measure residual gases in a vacuum system or to monitor changes in reactants or products in a process system.

Since the RGA is primarily used to measure the residual gases in the vacuum system and can measure the vacuum degree through analysis of the compositions of the residual gases while monitoring the amount of gas flowing into the vacuum system or chemical reaction occurring in the vacuum system in real time, the RGA is used for process monitoring of a semiconductor manufacturing process in the vacuum system.

Referring to FIG. 2, the RGA is capable of analyzing an analyte gas, which is coupled to a substrate processing apparatus 200 including a process chamber defining a processing space for substrate processing, and may include a mass analyzer 21 including an ion source (not shown), a controller 23 that analyzes the analyte gas based on data detected by the mass analyzer 21 and controls the mass analyzer 21, and a terminal 24 adapted to communicate with the controller 23.

The ion source (not shown) may be an electron impact ion source, which accelerates thermal electrons emitted upon application of electric current to a filament such that the thermal electrons can be ionized through collision with heavy elements or atoms.

The mass analyzer 21 may include a quadrupole filter, which is an electrode assembly composed of four parallel metal rods, and a detector.

In the RGA, vacuum pumps 22a, 22b may be coupled to the mass analyzer 21 to maintain a vacuum of 10-3 torr or less, which is an operating environment of the mass analyzer 21, and a valve V may be provided to an exhaust line FL of the substrate processing apparatus 200 to control a gas flow.

Despite advantages of no overlapping zones of each element and excellent sensitivity, since the RGA can suffer from significant deterioration in lifespan of a filament used as an ion source of the RGA when an analyte gas contains a corrosive gas, it is difficult for the RGA to be used in an environment with corrosive gases and to operate for a long period of time.

Referring to FIG. 1, typical gas analysis devices using self-plasma are coupled to a branch line branched from the exhaust line FL such that gases in the exhaust line FL can diffuse to flow into the gas analysis devices and the analyzed gases can be discharged back to the exhaust line FL along the branch line.

Even with a shut-off valve disposed on the branch line, since the shut-off valve is not a valve controlled in conjunction with operation of the gas analysis device, there is a problem in that the gas continuously flows through the branch line, increasing the contamination degree of the gas analysis device even when gas analysis is not required during the substrate processing process.

### SUMMARY OF INVENTION

### Technical Problem

It is one aspect of the present invention to provide a gas analysis device that has excellent sensitivity and resolution, can be operated for a long period of time even in an environment with contaminants or corrosive gases, and can monitor or diagnose a substrate processing process in real time without affecting the substrate processing process, and a substrate processing system comprising the same.

It is another aspect of the present invention to provide a gas analysis device, which includes a cleaning unit capable of cleaning contaminants accumulated in an orifice disposed therein using a laser such that the orifice can be cleaned without separation of the gas analysis device from the substrate processing system, thereby improving productivity of the substrate processing system without affecting continuity of the substrate processing process, and which can apply plasma mass spectrometry with high sensitivity and resolution to monitor/diagnose a substrate processing process that can cause significant contamination, and a substrate processing system comprising the same.

It is a further object of the present invention to provide a gas analysis device and a substrate processing system comprising the same, in which a first control valve is provided to a first connection pipe communicating with an ionizing unit to allow an analyte gas to flow into the ionizing unit and in which a controller controls the first control valve in conjunction with operation of the gas analysis device to prevent an analyte gas from unnecessarily flowing into the gas analysis device by closing the first control valve when gas analysis is unnecessary, thereby significantly reducing contamination of the gas analysis device caused by the analyte gas.

Technical Solution In accordance with one aspect of the present invention, there is provided a gas analysis device (100) disposed in a substrate processing system, the gas analysis device (100) including an ionizing unit (120) configured to produce ionized gas through ionization of an analyte gas introduced into the ionizing unit (120), a mass analysis unit (130) configured to analyze the mass of the ionized gas received from the ionizing unit (120), and a vacuum pump (140) coupled to the mass analysis unit (130) and adjusting an internal pressure of the mass analysis unit (130).

The gas analysis device (100) may further include a gas path orifice (150) disposed on an inflow path along which the analyte gas flows into the ionizing unit (120), and an ion path orifice (160) disposed on an outflow path along which the ionized gas flows out of the ionizing unit (120).

An internal pressure of the ionizing unit (120) may be maintained within a preset pressure range by the gas path orifice (150) and the ion path orifice (160).

The gas path orifice (150) may have a smaller diameter than the ion path orifice (160).

The gas path orifice (150) and the ion path orifice (160) may be coaxially located.

The gas analysis device (100) may further include a gas inflow chamber (110) disposed upstream of the ionizing unit (120) and formed with an inlet (110a) through which the analyte gas flows into the gas inflow chamber (110) and an outlet (110b) through which the analyte gas flows to the ionizing unit (120).

The gas path orifice (150) may be disposed at the inlet (110a) side of the gas inflow chamber (110).

A central axis of the gas path orifice (150) and a central axis of the ion path orifice (160) may be disposed to intersect each other at a point.

The central axis of the gas path orifice (150) and the central axis of the ion path orifice (160) may be disposed parallel to each other or in a skew position.

The gas inflow chamber (110) may be provided with a light transmissive third window (119).

The gas analysis device (100) may further include a spectroscopic analyzer (180) spectroscopically analyzing the analyte gas through the third window (119).

The gas analysis device (100) may further include an ion path orifice (160) disposed on an outflow path along which the ionized gas flows out of the ionizing unit (120) and a cleaning unit (170) configured to clean the ion path orifice (160) by emitting a laser beam toward the ion path orifice (160).

The cleaning unit (170) may include a first laser source and a first optical system directing a laser beam emitted from the first laser source toward the ion path orifice (160).

The gas analysis device (100) may further include a gas path orifice (150) disposed on an inflow path along which the analyte gas flows into the ionizing unit (120).

The gas path orifice (150) and the ion path orifice (160) may be coaxially located.

The first optical system may include a focus regulator regulating a focus of a laser beam such that the laser beam is focused on the gas path orifice (150) or the ion path orifice (160).

The gas analysis device (100) may further include a gas inflow chamber (110) disposed upstream of the ionizing unit (120) and formed with an inlet (110a) through which the analyte gas flows into the gas inflow chamber (110) and an outlet (110b) through which the analyte gas flows to the ionizing unit (120).

The gas path orifice (150) may be disposed at the inlet (110a) side of the gas inflow chamber (110).

The cleaning unit (170) may be disposed outside the gas inflow chamber (110).

The gas inflow chamber (110) may be provided with a first window (115) through which the laser beam emitted from the first laser source is transmitted.

A central axis of the gas path orifice (150) and a central axis of the ion path orifice (160) may be disposed to intersect each other at a point.

The gas inflow chamber (110) may be further provided with a second window (117) through which the laser beam emitted from the cleaning unit (170) is transmitted.

A central axis of the gas path orifice (150) and a central axis of the ion path orifice (160) may be disposed parallel to each other or in a skew position.

The gas inflow chamber (110) may be further provided with a second window (117) through which the laser beam emitted from the cleaning unit (170) is transmitted.

The cleaning unit (170) may further include a second laser source and a second optical system directing a laser beam emitted from the second laser source to travel toward the gas path orifice (150) through the second window (117).

The first optical system may include a beam splitter (172) splitting the laser beam emitted from the first laser source into two split beams and at least one reflection member (174) directing the two split beams split by the beam splitter (172) toward the ion path orifice (160) and the gas path orifice (150) through the first window (115) and the second window (117), respectively.

The first optical system may include a light path adjustment member adjusting a light path such that the laser beam emitted from the first laser source is selectively delivered to the gas path orifice (150) or the ion path orifice (160).

The cleaning unit (170) may further include a contamination detector detecting a contamination level of the ion path orifice (160).

The gas analysis device (100) may further include: a first control valve (CV1) provided to a first connection pipe (102) connected to the ionizing unit (120) to allow the analyte gas to flow into the ionizing unit (120); and a controller (190) controlling the first control valve (CV1) to be opened or closed.

The gas analysis device (100) may further include a second control valve (CV2) provide to a second connection pipe (104) through which the mass analysis unit (130) communicates with the exhaust line (FL).

The controller (190) may control the second control valve (CV2) to be opened or closed.

The gas analysis device (100) may be coupled to at least one of a process chamber defining a processing space for substrate processing, an exhaust line (FL) for discharging gas from the processing space to the outside, and a gas supply unit for supplying a process gas to the process chamber.

In accordance with another aspect of the present invention, there is provided a substrate processing system including: a substrate processing apparatus (200) including a process chamber defining a processing space for substrate processing; a gas supply unit for supplying a process gas to the process chamber, and the gas analysis device (100).

### Advantageous Effects

The gas analysis device according to the present invention and the substrate processing system comprising the same have excellent sensitivity and resolution, can operate for a long period time even in an environment with contaminants or corrosive gases, and can monitor or diagnose a substrate processing process in real time without affecting the process.

Specifically, the gas analysis device according to the present invention includes two orifices disposed at an inlet side, through which a gas flows into an ionization chamber adapted to form plasma, and at an outlet side, through which ionized gas flows out of the ionization chamber, to allow a mass spectrometer to operate in a high vacuum atmosphere and to allow the substrate processing process to be performed in a wide range of process pressures. With this structure, the gas analysis device can maintain the pressure of the ionization chamber in an appropriate range to stably generate/maintain plasma, can significantly reduce contamination of the gas analysis device through reduction in gas inflow amount, and allows plasma mass spectrometry securing excellent sensitivity and resolution to be applied to monitoring/diagnosis of the substrate processing process.

In addition, the gas analysis device according to the present invention includes a cleaning unit capable of cleaning contaminants accumulated in the orifices disposed inside the gas analysis device using a laser beam to perform cleaning of the orifices without separating the gas analysis device from the substrate processing system, thereby improving productivity of the substrate processing system without affecting continuity of the substrate processing process, and enabling application of plasma mass spectrometry with high sensitivity and resolution to monitoring/diagnosis of the substrate processing process that can cause significant contamination.

Furthermore, according to the present invention, a first control valve is provided to a first connection pipe communicating with an ionizing unit to allow an analyte gas to flow into the ionizing unit and is controlled to be opened or closed in conjunction with operation of the gas analysis device through a controller such that the first control valve can be closed to prevent the analyte gas from flowing into the gas analysis device when gas analysis is not required, thereby significantly reducing contamination of the gas analysis device caused by the analyte gas.

Furthermore, according to the present invention, a second control valve and a vacuum pump, which are controlled to be opened or closed, are provided to a second connection pipe, which connects a mass analysis unit and an exhaust line for communication therebetween, thereby forming a circulation structure that allows particles subjected to mass analysis to be discharged back into the exhaust line through the second connection pipe.

Furthermore, according to the present invention, the controller can control the first control valve and the second control valve to be opened or closed in conjunction with operation of the gas analysis device, thereby increasing a maintenance interval of the gas analysis device through reduction in contamination caused by gases entering the gas analysis device.

### BRIEF DESCRIPTION OF DRAWINGS

FIG. 1 is a conceptual diagram of a conventional SP-OES gas analysis device for analyzing an analyte gas of a substrate processing apparatus.

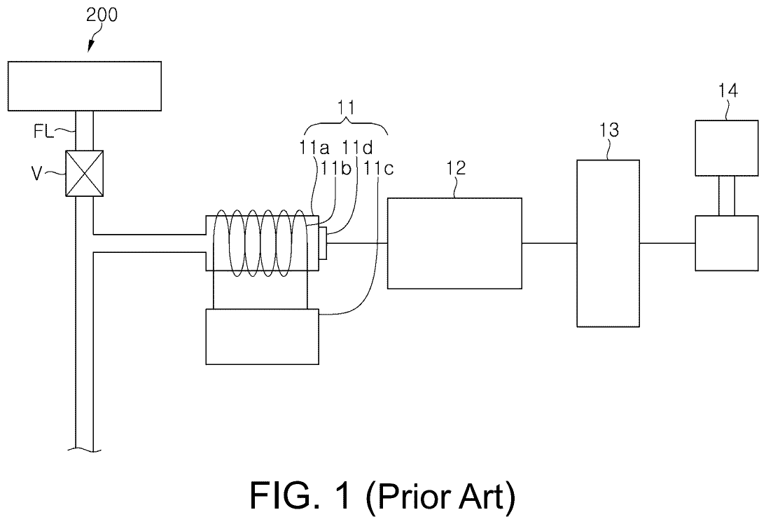

FIG. 2 is a conceptual diagram of a conventional RGA for analyzing an analyte gas of a substrate processing apparatus.

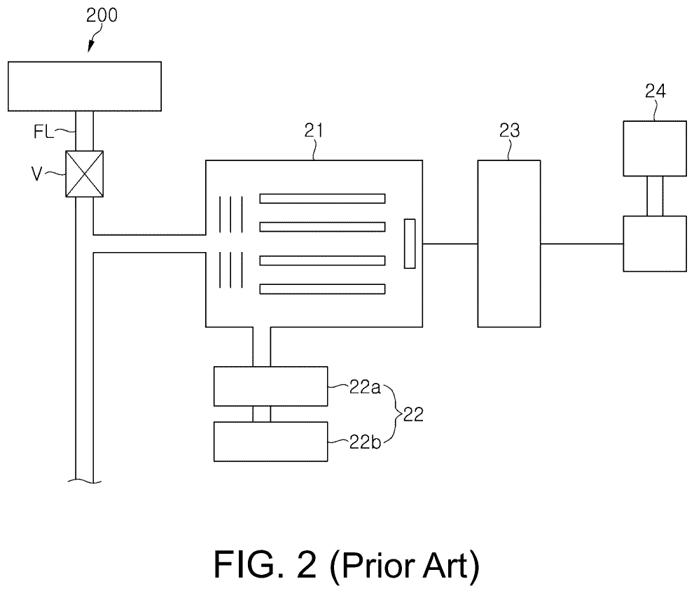

FIG. 3 is a conceptual diagram of a gas analysis device according to a first embodiment of the present invention and a substrate processing system including the same.

FIG. 4 is a conceptual diagram of a gas analysis device according to a second embodiment of the present invention and a substrate processing system including the same.

FIG. 5 is a conceptual diagram of a gas analysis device according to a third embodiment of the present invention and a substrate processing system including the same.

FIG. 6 is a conceptual diagram of a gas analysis device according to a fourth embodiment of the present invention and a substrate processing system including the gas analysis device.

FIG. 7 is a block diagram of a gas analysis device according to the present invention and a substrate processing system including the same.

### DESCRIPTION OF EMBODIMENTS

Hereinafter, exemplary embodiments of the present invention will be described in detail with reference to the accompanying drawings.

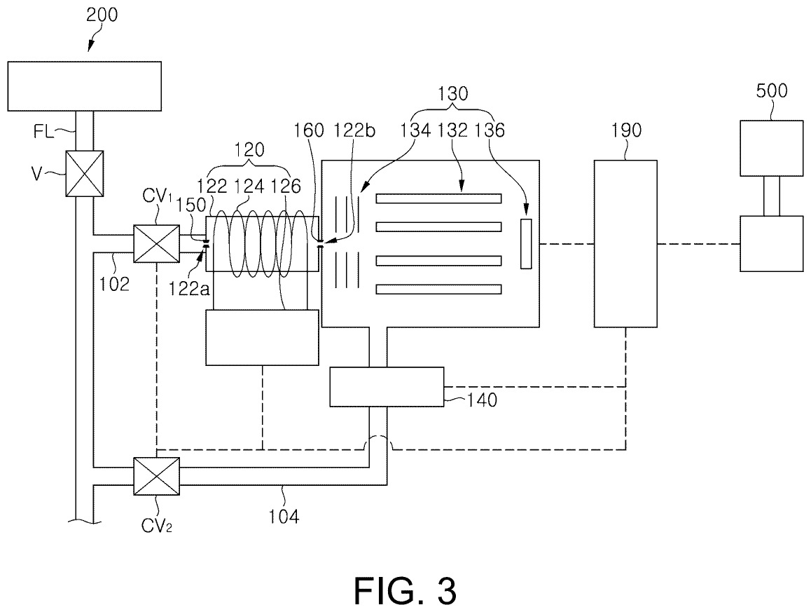

Referring to FIG. 3 to FIG. 7, a substrate processing system according to the present invention includes a substrate processing apparatus 200 including a process chamber defining a processing space for substrate processing; a gas supply unit for supplying a process gas to the process chamber; and a gas analysis device 100 for analyzing the analyte gas.

The substrate processing apparatus 200 may include the process chamber defining the processing space in which substrate processing, such as deposition, etching, and the like, is performed on a substrate, a substrate support disposed in the process chamber to support the substrate, and a gas injection unit disposed in the process chamber to inject a gas for substrate processing.

The process chamber defines the processing space for substrate processing, may have various configurations, and may have a cylindrical or hexahedral shape.

For example, the process chamber may include a chamber body open at an upper side thereof and an upper lid detachably coupled to an opening of the chamber body.

The chamber body is provided with the substrate support or the like, may have various configurations, and may be formed with at least one gate on an inner wall thereof to introduce and discharge the substrate into or from the processing space.

In addition, the chamber body may be formed with an exhaust port (not shown) through which gases or process products are exhausted from the processing space.

The exhaust port (not shown) may be coupled to an exhaust line FL that discharges gases from the processing space to the outside.

The exhaust line FL may be connected at one end thereof to a vacuum pump (not shown) to build up a pressure in the processing space to an appropriate process pressure (for example, a vacuum atmosphere).

As a target for substrate processing, the substrate is subjected to substrate processing, such as etching and deposition, and may include any substrate, such as a substrate for semiconductor manufacturing, a substrate for LCD manufacturing, a substrate for OLED manufacturing, a substrate for solar cell manufacturing, a transparent glass substrate, and the like.

The substrate support is disposed in the process chamber to support a substrate and may have various configurations.

The substrate support may be disposed at a lower side of the processing space in the process chamber and may include the substrate seating plate having a substrate seating surface on which the substrate is seated.

The substrate introduced into the process chamber by a transfer robot (not shown) may be seated on the substrate support to be chucked. To this end, the substrate seating plate may be provided with a vacuum chuck or an electrostatic chuck.

In addition, the substrate seating plate may further include a substrate temperature controller to control a temperature of the seated substrate to an appropriate process temperature. The substrate temperature controller is configured to heat or cool the substrate and may include a heating element or a coolant.

The gas injection unit is disposed in the process chamber to inject gas for substrate processing, may have various configurations, and may be connected to a system for supplying various process gases depending on the process.

For example, the gas may include a precursor, a reaction gas, a carrier gas, a purge gas, and the like as a process gas for deposition, etching, and the like and may include a corrosive gas containing Cl, F, H, N, or the like depending on the process, in which case a process by-product may also have corrosive properties.

Substrate processing, such as etching, deposition, lithography, and the like, performed by the substrate processing apparatus is not limited to specific physical or chemical processes, such as CVD, PVD, or ALD, and may include substrate processing processes using plasma, such as ICP (Inductively Coupled Plasma), CCP (Capacitively Coupled Plasma), ECR (Electron Cyclotron Resonance), and the like.

The substrate processing apparatus 200 may be an apparatus configured to perform substrate processing while maintaining a vacuum atmosphere or suppressing introduction of impurities.

Here, an internal pressure of the process chamber suitable for the substrate processing process may be set in various ways from vacuum to normal pressure depending on the kind of process and may be in the range of, for example, 0.01 torr to 10 torr depending on the type of process.

Process gases for substrate processing and by-products thereof can cause contamination or corrosion of the substrate processing system, in which case the process chamber may be made of corrosion-resistant materials and contamination caused by by-product particles can be cleaned by in-situ cleaning or remote plasma.

After substrate processing, process by-products or unreacted gases may be exhausted through the exhaust line FL.

The gas supply unit may include: a gas source supplying a process gas to the process chamber; and a gas supply line disposed between the gas source and the gas injection unit of the process chamber to deliver the process gas.

For the substrate processing apparatus 200, it is important to monitor and diagnose the substrate processing process in real time (for example, to determine an ending time of the process). Since many substrate processing processes, such as etching, CVD, and the like, generate a large amount of process by-products generating particles including polymers, which in turn are attached to the inner walls of the process chamber, and the like, there can be fluctuations in process parameters (process atmosphere, such as plasma and the like), which in turn cause generation of defects on the substrate during the process, causing deterioration in yield.

Accordingly, the substrate processing system according to the present invention include a gas analysis device 100 capable of analyzing gases to monitor the process and to diagnose process conditions in real time.

The gas analysis device 100 may be disposed in at least one of the process chamber defining a processing space for substrate processing, the exhaust line FL for discharging gas from the processing space to the outside, and the gas supply unit for supplying a process gas to the process chamber.

In a structure wherein the gas analysis device 100 is coupled to the process chamber, an analyte gas to be analyzed by the gas analysis device 100 may be a gas, which is received from the process chamber, in the processing space.

In a structure wherein the gas analysis device 100 is coupled to the exhaust line FL, the analyte gas may be a gas discharged to the exhaust line FL through the exhaust port (not shown) of the process chamber.

In a structure wherein the gas analysis device 100 is coupled to the gas supply unit, the analyte gas may be a process gas to be supplied to the process chamber.

The gas analysis device 100 may be disposed in the substrate processing system and may be a self-plasma mass spectrometer (SP-MS) that performs mass spectrometric analysis of ionized gas particles using plasma.

The gas analysis device 100 may include an ionizing unit 120 configured to produce ionized gas through ionization of an analyte gas introduced into the ionizing unit 120; a mass analysis unit 130 configured to analyze the mass of the ionized gas received from the ionizing unit 120; and a vacuum pump 140 coupled to the mass analysis unit 130 to adjust an internal pressure of the mass analysis unit 130.

The ionizing unit 120 may have various configurations and may produce ionized gas through ionization of the analyte gas introduced into the ionizing unit 120. Here, the ionized gas refers to plasma in which the analyte gas is ionized into particles.

For example, the ionizing unit 120 may be a plasma generation module capable of generating plasma. Since the ionizing unit 120 can generate self-plasma, the gas analysis device 100 according to the present invention can also perform monitoring or diagnosis of substrate processing processes that do not use plasma.

Specifically, referring to FIG. 3 to FIG. 6, the ionizing unit 120 may include an ionization chamber 122 defining an internal space in which an analyte gas is ionized, an electrode 124 that generates an induced electric field for ionization in the internal space of the ionization chamber 122, and an RF power source 126 that applies RF power to the electrode 124.

The ionization chamber 122 refers to a chamber in which the analyte gas is ionized, and may be formed in various configurations using a corrosion resistant material, such as ceramics, quartz, sapphire, and the like, to allow continuous operation for a long period of time even in a corrosive environment.

The electrode 124 may have various configurations to form an inductive electric field for ionization of the analyte gas in the internal space of the ionization chamber 122.

For example, the electrode 124 may be a coil (antenna) wound around an outer circumferential surface of the ionization chamber 122 and the induced electric field generated by the electrode 124 may provide energy for ionizing the analyte gas in the internal space of the ionization chamber 122.

The RF power source 126 is a power source that applies RF power of a preset frequency to the electrode 124, and may have various configurations. For example, the RF power source 126 may include a power source that applies an RF power of 50 MHz, a matcher for impedance matching, and a voltage monitor that monitors the applied voltage.

Further, the RF power source 126 may further include an igniter for plasma ignition.

When RF power is applied to the electrode 124 by the RF power source 126, an induced electric field can be generated in the ionization chamber 122 and can ionize the analyte gas to produce ionized gas (plasma).

In order for the plasma generated within the ionization chamber 122 to remain stable, it is desirable that the ionization chamber 122 maintain an appropriate internal pressure. An appropriate internal pressure for the plasma to remain stable within the ionization chamber 122 is at least 10-3 torr, more preferably in the range of 1 torr to 10-2 torr.

As the ionization chamber 122 maintains a lower internal pressure than the “process chamber, the exhaust line FL of the substrate processing apparatus 200, or the gas supply unit”, the analyte gas can be introduced into the ionization chamber 122.

The ionization chamber 122 is a hollow chamber (with a circular or square shape) having a certain length and defining an internal space therein, and may be formed at both ends thereof with an inlet 122a through which an analyte gas flows into the ionization chamber 122 and an outlet 122b through which the ionized gas flows out of the ionization chamber 122.

The inlet 122a may communicate with “the process chamber, the exhaust line FL of the substrate processing apparatus 200, or the gas supply unit” to allow the analyte gas to be introduced into the ionization chamber 122 through the inlet 122a.

The outlet 122b is an opening for outflow of the ionized gas from the ionization chamber 122 and the ionized gas discharged through the outlet 122b may be introduced into the mass analysis unit 130 described below.

The inlet 122a and the outlet 122b may be located on a straight line parallel to a longitudinal direction of the ionization chamber 122. Here, a central axis passing through a center of the inlet 122a may coincide with a central axis passing through a center of the outlet 122b.

The ionizing unit 120 may communicate with the “process chamber, exhaust line (FL), or gas analysis unit” through the first connection pipe 102. The analyte gas may flow into the inlet 122a of the ionization chamber 122 through the first connection pipe 102.

The first connection pipe 102 is configured to connect the ionizing unit 120 to the “process chamber, exhaust line (FL), or gas analysis unit” to each other for communication therebetween and may be provided with a first control valve CV1 that is controlled to be opened or closed. The first connection pipe 102 may be further provided with a pressure sensor P that senses a pressure in the first connection pipe 102.

The first control valve CV1 may be selected from various valves including a gate valve, a ball valve, a butterfly valve, a cock valve, a diaphragm valve, and the like so long as opening and closing of the valve can be controlled.

The mass analysis unit 130 may analyze the mass of the ionized gas received from the ionizing unit 120 and may have various configurations.

The mass analysis unit 130 may be capable of measuring the masses of ions constituting the ionized gas in a mass-to-charge ratio and may include a filter capable of separating the ionized gas based on the mass-to-charge ratio.

Specifically, the mass analysis unit 130 may be a quadrupole mass spectrometer and may include a quadrupole filter 132, an ion optic 134 that delivers the ionized gas received from the ionizing unit 120 to the quadrupole filter 132, and a detector 136 that detects a signal generated by ions having passed through the quadrupole filter 132.

Since the quadrupole filter 132 is composed of four parallel metal bars and a voltage applied to each metal bar affects a traveling path of ions passing therethrough such that only ions having a certain mass-to-charge ratio at an applied voltage travel along the traveling path while other ions deviate from the traveling path, a mass spectrum can be obtained by measuring the ions passing through the quadrupole filter 132 at various voltages. The principle of the quadrupole mass spectrometer is well known in the art and will not be described in detail.

The ion optic 134 is disposed upstream of the quadrupole filter 132 to exclude unnecessary particles before delivering the ionized gas to the quadrupole filter 132.

Although the analyte gas is ionized in the ionization chamber 122, electrons and neutral particles are mixed together with analyte ions in the ionization chamber 122, the ion optic 134 allows the analyte ions to be introduced into the quadrupole filter 132 as much as possible while preventing unnecessary electrons and neutral particles from being introduced into the quadrupole filter 132. As a result, it is possible to improve resolution and sensitivity of the gas analysis device while reducing noise.

The detector 136 may have various configurations to detect signals generated by ions having passed through the quadrupole filter 132.

After passing through the quadrupole filter 132, the ions enter the detector 136, which detects signals produced by the captured ions to derive a mass spectrum. The detector 136 may be, for example, an electron multiplier, a Faraday cup, or a secondary electron multiplier (SEM), without being limited thereto.

On the other hand, since it is ideal that the analyte ions (cations) move to the detector 136 only under the influence of an electric field, the mass analysis unit 130 operates in a high-vacuum atmosphere to avoid collision between particles. More specifically, the mass analysis unit 130 is maintained at a maximum internal pressure of 10-3 torr or less and efficiently operates at 10-4 torr or less.

To this end, the gas analysis device 100 includes a vacuum pump 140 coupled to the mass analysis unit 130 to adjust the internal pressure of the mass analysis unit 130.

The vacuum pump 140 may include a turbo molecular pump 140a, which is a high vacuum pump to maintain a low vacuum in the mass analysis unit 130, and may further include a backing pump 140b to assist the turbo molecular pump.

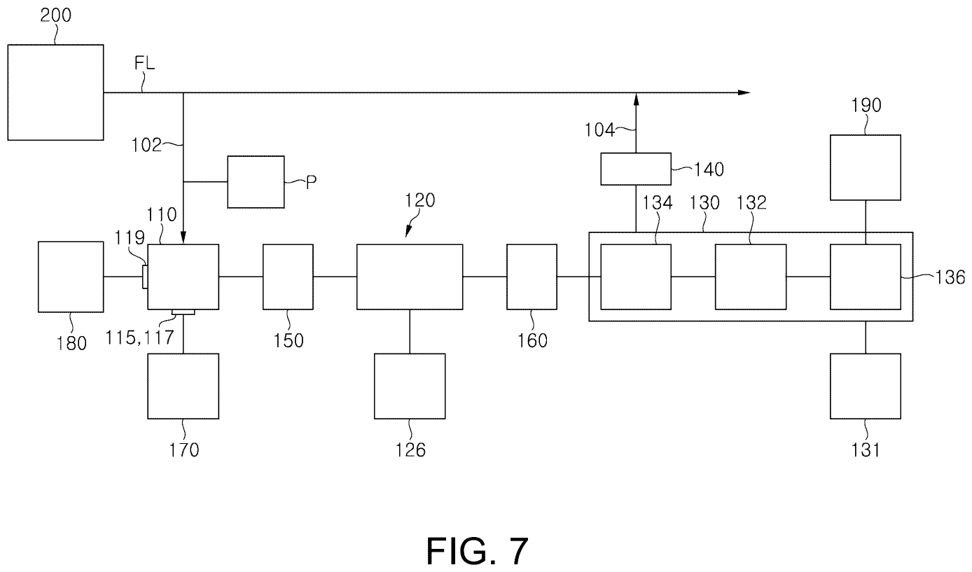

In addition, the mass analysis unit 130 may further include a pressure sensor 131 to detect vacuum degree, as shown in FIG. 7.

Since the mass analysis unit 130 has a high vacuum internal pressure created by the vacuum pump 140, the ionized gas can flow into the mass analysis unit 130 from the ionization chamber 122, in which a relatively high internal pressure is created.

The mass analysis unit 130 may communicate with the exhaust line FL through the second connection pipe 104. Particles in the mass analysis unit 130 may flow into the exhaust line FL through the second connection pipe 104.

The second connection pipe 104 connects the mass analysis unit 130 to the exhaust line FL for communication therebetween and is connected to a more downstream side of the exhaust line FL than the first connection pipe 102. The second connection pipe 104 may be provided with a second control valve CV2, which is controlled to be opened or closed.

The second control valve CV2 may be selected from various valves including a gate valve, a ball valve, a butterfly valve, a cock valve, a diaphragm valve, and the like so long as opening and closing of the valve can be controlled.

In addition, the second connection pipe 104 may be provided with the vacuum pump 140 as described above.

The gas analysis device 100 including the above components may further include a controller 190 that controls operation of the gas analysis device 100.

The controller 190 may control opening/closing of the first control valve CV1 and, when the gas analysis device 100 further includes the second control valve CV2, the controller 190 may also control opening/closing of the second control valve CV2.

The controller 190 may control the overall operation of the substrate processing system or may control the operation of the gas analysis device 100 in response to control signals from a main controller of the substrate processing system.

In some cases, monitoring or diagnosis of the substrate processing process does not need to be continuously performed during the substrate processing process. Although it is sufficient that gas analysis is performed only for a certain period of time during the substrate processing process, there is a problem that contamination of the gas analysis device 100 is accelerated, if a large amount of analyte gas continuously flows into the gas analysis device 100 during the substrate processing process.

Since large amounts of analyte particles continuously flow into the internal space of the protrusion from the enclosure, Korean Patent Publication No. 10-2008-0019279, which is disclosed in the background, also has the same problem of contamination.

According to the present invention, the first control valve CV1 is provided to the first connection pipe 102, and the controller 190 controls the first control valve CV1 to be opened or closed according to operation of the gas analysis device 100 (performing or stopping gas analysis) and may close the first control valve CV1 to prevent the analyte gas from flowing into the gas analysis device 100 when gas analysis is not required, thereby reducing contamination of the gas analysis device 100.

Furthermore, in a conventional gas analysis device, the gas introduced into the gas analysis device is not exhausted, causing contamination of the gas analysis device by residual particles, whereas the gas analysis device according to the present invention exhausts the particles in the mass analysis unit 130 to the exhaust line FL through the second connection pipe 104, thereby minimizing contamination of the gas analysis device 100.

The controller 190 may control not only the first control valve CV1 and the second control valve CV2, but also the ionizing unit 120, the mass analysis unit 130, and the cleaning unit 170 described below.

On the other hand, the vacuum degree of the ionization chamber 122 for normal operation of the gas analysis device 100 is different from the vacuum degree of the mass analysis unit 130. Since the mass analysis unit 130 requires that the internal pressure be maintained at a maximum of 10-3 torr or less and the mass analysis unit 130 efficiently operates at 10-4 torr or less, whereas the ionization chamber 122 efficiently operates in a higher pressure range (at least 10-3 torr, more preferably in the range of 1 torr to 10-2 torr), it is desirable that the internal pressure conditions also be maintained stable for stable plasma formation within the ionization chamber 122.

Although the mass analysis unit 130 can have a high vacuum atmosphere created and maintained by the vacuum pump 140, the ionization chamber 122 communicates with the “process chamber, exhaust line (FL), or gas analysis unit” and the mass analysis unit 130 and does not have a separate pump for pressure control. Thus, there is a need for a means that maintains the pressure within the ionization chamber 122 in an appropriate range.

In particular, since the process chamber has a wide range of pressures between 10-2 torr and 10 torr depending on the type of process, it is essential that an appropriate vacuum level of the ionization chamber 122 is stably maintained in order to use the gas analysis device 100 for a long period of time (continuous use for 3 months or more) over a wide range of process pressures.

To this end, the gas analysis device 100 according to the present invention includes a gas path orifice 150 and an ion path orifice 160 disposed on an inflow path along which the analyte gas flows into the ionizing unit 120 and on an outflow path along which the ionized gas flows out of the ionizing unit 120, respectively.

The gas path orifice 150 may be disposed on the inflow path along which the analyte gas flows into the ionization chamber 122.

The gas path orifice 150 refers to a plate having a small orifice formed therein and disposed on the inflow path along which the analyte gas flows into the ionization chamber 122 of the ionizing unit 120. The orifice may be a cylindrical opening having the same diameter in a flow direction of the analyte gas or a conical opening having a diameter gradually increasing or decreasing in the flow direction of the analyte gas, and may have various shapes, without being limited thereto.

The orifice formed in the gas path orifice 150 may be present singularly or in plural.

Furthermore, the gas path orifice 150 may be provided in plural and a plurality of gas path orifices 150 may be spaced apart from each other to form a multistage structure. The size of the orifices formed in the plurality of gas path orifices 150 may be the same or may be variable in an inflow direction of the analyte gas.

When the gas path orifice 150 is provided in plural and is realized as a combination of orifice plates each having multiple orifices, the centers of the orifices formed in the plurality of gas path orifices 150 may be coaxially aligned, without being limited thereto.

Furthermore, a valve controlled to be opened or closed (by an electrical signal) may be further disposed on the flow path on which the gas path orifices 150 are disposed.

For example, the valve may be disposed upstream of the gas path orifice 150 such that the degree of opening or closing is controlled, thereby regulating the gas flow rate through the gas path orifice 150.

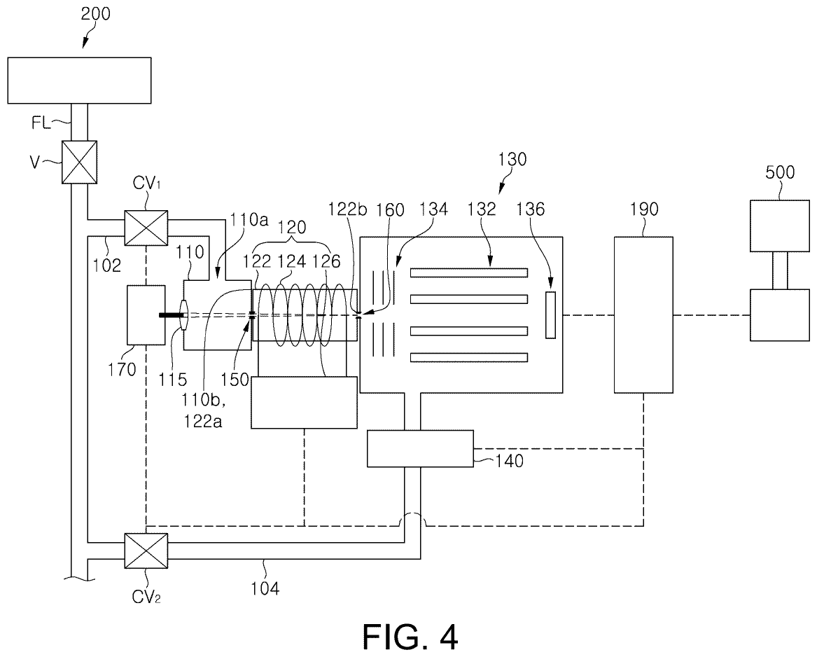

In one embodiment, referring to FIG. 3 and FIG. 4, the gas path orifice 150 may be disposed at the inlet 122a side of the ionization chamber 122 in the inflow path of the analyte gas.

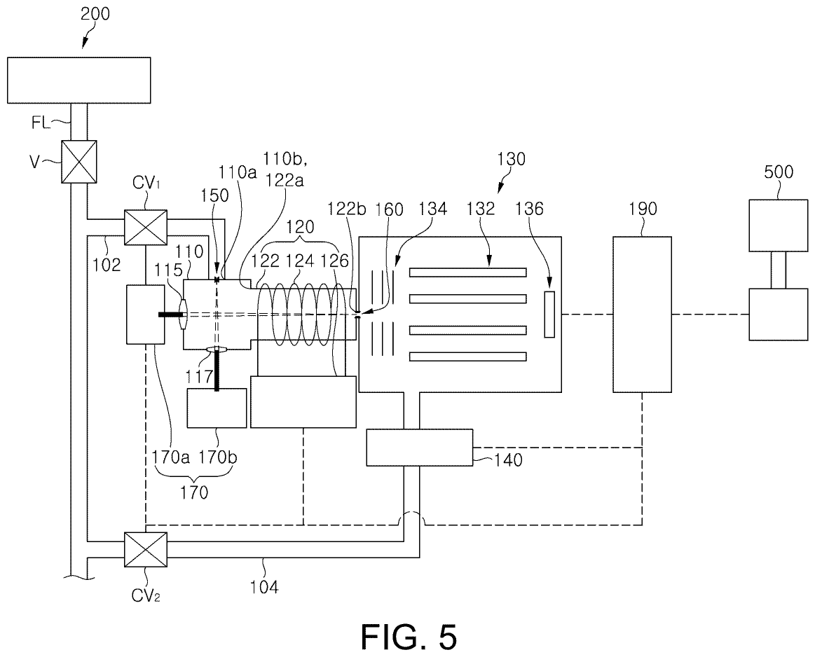

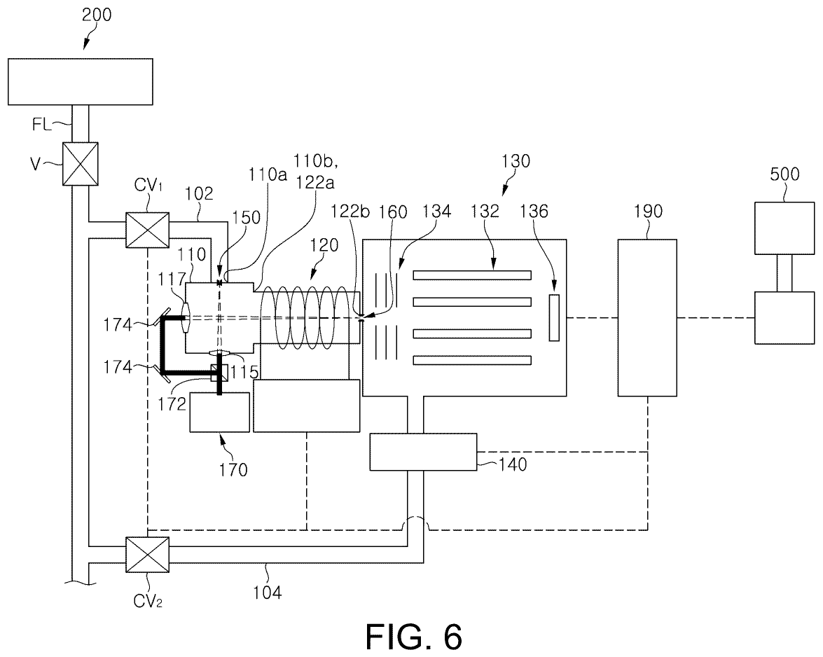

In another embodiment, referring to FIG. 5 and FIG. 6, the gas analysis device 100 may further include a gas inflow chamber 110 disposed upstream of the ionizing unit 120, in which the gas path orifice 150 may be disposed at an inlet 110a side of the gas inflow chamber 110.

The gas inflow chamber 110 is formed with an inlet 110a through which the analyte gas flows from the “process chamber, exhaust line (FL), or gas supply” into the gas inflow chamber and an outlet 110b through which the analyte gas flows to the ionizing unit 120, and may have various configurations.

The gas inflow chamber 110 may be formed in various shapes so long as a space for the analyte gas to flow is defined therein. Like the ionization chamber 122, the gas inflow chamber 110 may be formed of a corrosion-resistant material, such as ceramics, quartz, sapphire, and the like to allow continuous operation for a long period of time in a corrosive environment.

The gas inflow chamber 110 may be disposed upstream of the ionization chamber 122 such that the introduced analyte gas is delivered to the ionization chamber 122.

The gas inflow chamber 110 is formed with the inlet 110a through which the analyte gas flows to the gas inflow chamber 110 and with the outlet 110b through which the analyte gas flows to the ionizing unit 120.

The gas path orifice 150 may be disposed at the outlet 110b side of the gas inflow chamber 110, as shown in FIG. 3 or FIG. 4, or at the inlet 110a side of the gas inflow chamber 110, as shown in FIG. 5 or FIG. 6.

Although not shown in the drawings, it is obvious that that the gas path orifice 150 may be disposed within the first connection pipe 102 upstream of the gas inflow chamber 110 or may be disposed in a separate flow path between the gas inflow chamber 110 and the ionizing unit 120.

The ion path orifice 160 may be disposed on an outflow path of the ionized gas from the ionizing unit 120.

The ion path orifice 160 refers to a plate having a small orifice formed therein and disposed on the outflow path along which the ionized gas flows out of the ionization chamber 122 of the ionizing unit 120. The orifice may be a cylindrical opening having the same diameter in the flow direction of the analyte gas or a conical opening having a diameter gradually increasing or decreasing in the flow direction of the analyte gas, and may have various shapes, without being limited thereto.

The orifice formed in the ion path orifice 160 may be present singularly or in plural.

Furthermore, the ion path orifice 160 may be provided in plural and a plurality of ion path orifices 160 may be spaced apart from each other to form a multistage structure. The size of the orifices formed in the plurality of ion path orifices 160 may be the same or may be variable in an inflow direction of ions.

The centers of the orifices formed in the plurality of ion path orifices 160 may be coaxially aligned, without being limited thereto.

When the ion path orifice 160 is provided in plural and is realized as a combination of orifice plates having multiple orifices, the centers of the orifices formed in the plurality of ion path orifices 160 may be coaxially aligned, without being limited thereto.

Furthermore, a valve controlled to be opened or closed (by an electrical signal) may be further disposed on the flow path on which the ion path orifices 160 are disposed.

For example, the valve may be disposed upstream of the ion path orifice 160 such that the degree of opening or closing is controlled, thereby regulating the ion flow rate through the ion path orifice 160.

In addition, the plurality of ion path orifices 160 may act as ion optics by applying a voltage.

In one embodiment, referring to FIG. 3 to FIG. 6, the ion path orifice 160 may be disposed at the outlet 122b side of the ionization chamber 122.

Although FIG. 3 to FIG. 6 illustrate an example in which the ion path orifice 160 is disposed on the outlet 122b side of the ionizing unit 120, it is obvious that the ion path orifice 160 is disposed in a separate flow path between the ionizing unit 120 and the mass analysis unit 130 or is disposed at the inlet side of the mass analysis unit 130.

The analyte gas flows in and the ionized gas flows out through the gas path orifice 150 and the ion path orifice 160, respectively, with the ionization chamber 122 disposed therebetween, whereby the pressure inside the ionization chamber 122 can be stably maintained within a predetermined pressure range during operation.

In addition, the typical gas analysis device does not include the gas path orifice 150 and thus has a problem in that a large amount of gas flows into the gas analysis device, causing increase in contamination level, whereas the gas analysis device according to the present invention includes the gas path orifice 150, thereby significantly reducing contamination of the gas analysis device 100 through significant reduction in the amount of analyte gas flowing into the gas analysis device 100.

Here, the orifice of the gas path orifice 150 may have a smaller diameter than the orifice of the ion path orifice 160. If the size of the orifice of the ion path orifice 160 becomes excessively small, the sensitivity of the mass analysis unit 130 can be reduced and the orifice can be easily blocked even by small contaminants. Thus, it is necessary to maintain the size of the ion path orifice 160 at a certain size or larger.

The size of the gas path orifice 150 may be set in consideration of a suitable internal pressure of the ionization chamber 122.

Referring to FIG. 3 and FIG. 4, the gas path orifice 150 and the ion path orifice 160 may be coaxially located.

The central axis of the gas path orifice 150 may coincide with the central axis of the ion path orifice 160 and may be parallel to the longitudinal direction of the ionization chamber 122.

In another embodiment, referring to FIG. 5 and FIG. 6, the central axis of the gas path orifice 150 and the central axis of the ion path orifice 160 may be disposed to intersect each other at a point. Preferably, the central axis of the gas path orifice 150 and the central axis of the ion path orifice 160 may intersect perpendicularly.

In another embodiment, the central axis of the gas path orifice 150 and the central axis of the ion path orifice 160 may be disposed parallel to each other or in a skew position.

Since the gas path orifice 150 and the ion path orifice 160 are formed with small orifices, the gas path orifice 150 and the ion path orifice 160 can be contaminated and blocked during long-term use, causing difficulty in normal operation. In order to clean or replace the contaminated gas path orifice 150 and the contaminated ion path orifice 160 with new ones, the gas analysis device 100 must be separated from the substrate processing apparatus and reinstalled thereto, thereby causing a problem of affecting continuity of the substrate processing process.

Accordingly, the gas analysis device 100 according to the present invention further includes a cleaning unit 170 that cleans the gas path orifice 150 and the ion path orifice 160.

The cleaning unit 170 can remove (sublimate, evaporate) contaminants deposited on the gas path orifice 150 and the ion path orifice 160 by emitting a laser beam to the gas path orifice 150 and the ion path orifice 160.

The wavelength of the laser beam may be adjusted to remove only surface contaminants without affecting the gas path orifice 150 and the ion path orifice 160.

In a structure wherein the gas analysis device 100 includes only the ion path orifice 160, the cleaning unit 170 may act as a cleaning means for configured to clean the ion path orifice 160.

The cleaning unit 170 may include a first laser source and a first optical system that directs a laser beam emitted from the first laser source towards the ion path orifice 160.

The laser beam emitted from the first laser source may be focused on the ion path orifice 160 through the first optical system. The first optical system may have various configurations to form a light path for the emitted laser beam and may include at least one lens or reflection member.

For the gas analysis device 100 further provided with the gas path orifice 150 on the inflow path along which the analyte gas flows into the ionizing unit 120, as shown in FIG. 3 to FIG. 6, it is obvious that the gas path orifice 150 may also be cleaned by the cleaning unit 170.

In a structure wherein the gas path orifice 150 and the ion path orifice 160 are coaxially disposed, the first optical system may include a focusing unit to adjust the focus of the laser beam such that the laser beam is focused on the gas path orifice 150 or the ion path orifice 160.

In a structure wherein the gas analysis device 100 includes the gas inflow chamber 110, the cleaning unit 170 may be disposed outside the gas inflow chamber 110.

In this structure, the gas inflow chamber 110 may be provided with a first window 115 through which the laser beam emitted from the cleaning unit 170 can be transmitted.

After passing through the first window 115, the laser beam may be focused on the gas path orifice 150 and the ion path orifice 160 to clean the gas path orifice 150 and the ion path orifice 160.

FIG. 4 illustrates an embodiment configured to clean both the gas path orifice 150 and the ion path orifice 160 using a single first laser source and a single first optical system.

On the other hand, as shown in FIG. 5 to FIG. 6, in the structure wherein the gas path orifice 150 is disposed at the inlet 110a side of the gas inflow chamber 110 such that the central axis of the gas path orifice 150 and the central axis of the ion path orifice 160 intersect each other at a point, the gas inflow chamber 110 may be further provided with a second window 117 through which the laser beam emitted from the cleaning unit 170 can be transmitted.

Furthermore, even in the structure wherein the gas path orifice 150 is disposed at the inlet 110a side of the gas inflow chamber 110 such that the central axis of the gas path orifice 150 and the central axis of the ion path orifice 160 are arranged parallel to each other or in a skew position, the gas inflow chamber 110 may be further provided with the second window 117 through which the laser beam emitted from the cleaning unit 170 can be transmitted.

Referring to FIG. 5, in this structure, the cleaning unit 170 may further include a second laser source and a second optical system that directs a laser beam emitted from the second laser source to travel toward the gas path orifice 150 through the second window 117. The second optical system may have various configurations to form a traveling path of the laser beam and may include at least one lens or reflection member.

That is, the cleaning unit 170 may be separately provided with a first cleaning unit 170a, which includes the first laser source and the first optical system, and a second cleaning unit 170b, which includes the second laser source and the second optical system.

The first cleaning unit 170a serves to clean the ion path orifice 160 and the laser beam emitted from the first cleaning unit 170a may be focused on the ion path orifice 160 through the first window 115.

The second cleaning unit 170b serves to clean the gas path orifice 150 and the laser beam emitted from the second cleaning unit 170b may be focused on the gas path orifice 150 through the second window 117.

The first cleaning unit 170a and the second cleaning unit 170b may be controlled and operated independently of each other by the controller 190.

Further, the cleaning unit 170 may include a single first laser source and may split the emitted laser beam into two split beams to clean the gas path orifice 150 and the ion path orifice 160, as shown in FIG. 6.

To this end, the first optical system may include a beam splitter 172 that splits a laser beam emitted from the first laser source into two split beams, and at least one reflection member 174 that directs the two split beams split by the beam splitter 172 to travel toward the ion path orifice 160 and the gas path orifice 150 through the first window 115 and the second window 117, respectively.

The two split beams split by the beam splitter 172 may pass through the first window 115 and the second window 117 to be delivered to the ion path orifice 160 and the gas path orifice 150, respectively.

In the structure wherein the cleaning unit 170 includes a single first laser source, as shown in FIG. 4 and FIG. 6, the gas path orifice 150 and the ion path orifice 160 may be cleaned simultaneously or sequentially in a time-slicing manner. Simultaneous cleaning of the two flow orifice plates 150, 160 using a single first laser source requires a high power light source, whereas time-slicing cleaning of the two flow orifice plates 150, 160 allows application of a relatively low power laser source.

When the gas path orifice 150 and the ion path orifice 160 are subjected to time-slicing cleaning using a single first laser source, the first optical system of the cleaning unit 170 may include a light path adjustment member for adjusting a light path such that a laser beam emitted from the first laser source is selectively delivered to the gas path orifice 150 or the ion path orifice 160.

The light path adjustment member may have various configurations capable of adjusting the light path such that the laser beam emitted from the first laser source is selectively delivered to the gas path orifice 150 or the ion path orifice 160, and may include, for example, a rotatable mirror scanner capable of adjusting a traveling direction of reflected light.

Cleaning timing for the gas and ion path orifices 150, 160 may be determined in various ways.

For example, the cleaning unit 170 may perform cleaning of the flow orifice plates 150, 160 at preset time intervals or at preset points in time.

In another embodiment, the cleaning unit 170 may further include a contamination detector that detects a contamination level of the ion path orifice 160.

The contamination detector may have various configurations capable of detecting whether the orifice of the ion path orifice 160 is blocked by contaminants, and may include, for example, a sensor that detects the amount of ionized gas passing through the ion path orifice 160.

The contamination detector may also be configured to detect a contamination level of the gas path orifice 150.

An initiation time for cleaning may be determined by detecting the contamination level of the ion path orifice 160 (or the gas path orifice 150) through the contamination detector. That is, a cleaning process may be initiated when the detected contamination level exceeds a preset standard.

For example, when the contamination levels of the gas and ion path orifices 150, 160 detected by the gas analysis device 100 exceeds preset standards, the controller 190 may close the first control valve CV1 and the second control valve CV2 and stop operation of the ionizing unit 120 and the mass analysis unit 130 while initiating operation of the cleaning unit 170 to start a cleaning process for the gas and ion path orifices 150, 160.

The controller 190 may control the cleaning time, cleaning duration, and cleaning interval of the cleaning unit 170 to ensure effective cleaning without affecting the substrate processing process.

The gas analysis device 100 may further include a spectroscopic analyzer 180 that spectroscopically analyzes the analyte gas.

The spectroscopic analyzer 180 may be an OES (Optical Emission Spectrometer) including a spectroscopic sensor to detect a light spectrum of an analyte gas and may send a detected signal to the controller 190.

To this end, the gas inflow chamber 110 of the gas analysis device 100 may be further provided with a light transmissive third window 119.

The third window 119 is disposed so as not to interfere with the first window 115 and the second window 117 and the spectroscopic analyzer 180 may perform spectroscopic analysis on light having passed through the third window 119.

The controller 190 may control the gas analysis device 100 based on a detection value detected from the gas analysis device 100 while communicating with a terminal 500 (personal computer and the like) on which software (SW) for operation of a substrate processing system is installed.

The detection values from the gas analysis device 100 may include various measurement data, such as mass spectra of ionized gases, the contamination levels of the gas and ion path orifices 150, 160, internal pressures, and the like.

Although FIG. 3 to FIG. 7 illustrate the embodiment in which the gas analysis device 100 according to the present invention is disposed in the exhaust line FL, it should be understood that the present invention is not limited thereto and may also include embodiments in which the gas analysis device 100 is coupled to the process chamber, the exhaust line FL, or the gas supply unit for supplying a process gas to the process chamber.

Although some embodiments have been described herein, it should be understood that these embodiments are provided for illustration only and are not to be construed in any way as limiting the present invention, and that various modifications and changes can be made by those skilled in the art without departing from the spirit and scope of the invention.

1. A gas analysis device installed in a substrate processing system, the gas analysis device comprising:
an ionizing unit configured to produce ionized gas through ionization of an analyte gas introduced into the ionizing unit; a mass analysis unit configured to analyze the mass of the ionized gas received from the ionizing unit; and a vacuum pump coupled to the mass analysis unit to adjust an internal pressure of the mass analysis unit.

2. The gas analysis device according to claim 1, further comprising:
a gas path orifice disposed in an inflow path along which the analyte gas flows into the ionizing unit; and an ion path orifice disposed in an outflow path along which the ionized gas flows out of the ionizing unit,
wherein an internal pressure of the ionizing unit is maintained within a preset pressure range by the gas path orifice and the ion path orifice.

3. The gas analysis device according to claim 2, wherein the gas path orifice has a smaller diameter than the ion path orifice.

4. The gas analysis device according to claim 2, wherein the gas path orifice and the ion path orifice are coaxially located.

5. The gas analysis device according to claim 2, further comprising:
a gas inflow chamber disposed upstream of the ionizing unit and formed with an inlet through which the analyte gas flows into the gas inflow chamber and an outlet through which the analyte gas flows to the ionizing unit,
wherein the gas path orifice is disposed at the inlet side of the gas inflow chamber.

6. (canceled)

7. (canceled)

8. The gas analysis device according to claim 5, wherein
the gas inflow chamber is provided with a light transmissive third window,
the gas analysis device further comprising a spectroscopic analyzer spectroscopically analyzing the analyte gas through the third window.

9. The gas analysis device according to claim 1, further comprising:
an ion path orifice disposed on an outflow path along which the ionized gas flows out of the ionizing unit; and
a cleaning unit configured to clean the ion path orifice by emitting a laser beam toward the ion path orifice.

10. The gas analysis device according to claim 9, wherein the cleaning unit includes a first laser source and a first optical system configured to direct a laser beam emitted from the first laser source toward the ion path orifice.

11. The gas analysis device according to claim 10, further comprising:
a gas path orifice disposed on an inflow path along which the analyte gas flows into the ionizing unit.

12. The gas analysis device according to claim 11, wherein
the gas path orifice and the ion path orifice are coaxially located, and
the first optical system includes a focus regulator configured to regulate a focus of a laser beam such that the laser beam is focused on the gas path orifice or the ion path orifice.

13. The gas analysis device according to claim 11, further comprising:
a gas inflow chamber disposed upstream of the ionizing unit and formed with an inlet through which the analyte gas flows into the gas inflow chamber and an outlet through which the analyte gas flows to the ionizing unit,
wherein the gas path orifice is disposed at the inlet side of the gas inflow chamber.

14. The gas analysis device according to claim 13, wherein
the cleaning unit is disposed outside the gas inflow chamber, and
the gas inflow chamber is provided with a first window through which the laser beam emitted from the first laser source is transmitted.

15. The gas analysis device according to claim 14, wherein
the gas inflow chamber is further provided with a second window through which the laser beam emitted from the cleaning unit is transmitted.

16. (canceled)

17. The gas analysis device according to claim 15, wherein the cleaning unit further includes a second laser source and a second optical system configured to direct a laser beam emitted from the second laser source to travel toward the gas path orifice through the second window.

18. The gas analysis device according to claim 15, wherein the first optical system includes a beam splitter splitting the laser beam emitted from the first laser source into two split beams, and at least one reflection member directing the two split beams split by the beam splitter toward the ion path orifice and the gas path orifice through the first window and the second window, respectively.

19. The gas analysis device according to claim 15, wherein the first optical system includes a light path adjustment member adjusting a light path such that the laser beam emitted from the first laser source is selectively delivered to the gas path orifice or the ion path orifice.

20. The gas analysis device according to claim 9, wherein the cleaning unit further comprises a contamination detector configured to detect a contamination level of the ion path orifice.

21. The gas analysis device according to claim 1, further comprising:
a first control valve provided to a first connection pipe connected to the ionizing unit to allow the analyte gas to flow into the ionizing unit; and
a controller controlling the first control valve to be opened or closed.

22. The gas analysis device according to claim 21, further comprising:
a second control valve provide to a second connection pipe through which the mass analysis unit communicates with the exhaust line,
wherein the controller controls the second control valve to be opened or closed.

23. The gas analysis device according to claim 1, wherein the gas analysis device is coupled to at least one of a process chamber defining a processing space for substrate processing, an exhaust line for discharging gas from the processing space to the outside, and a gas supply unit for supplying a process gas to the process chamber.

24. (canceled)
